(19) United States
(12) Patent Application Publication
(10) Pub. No.: US 2011/0054449 A1
(43) Pub. Date: Mar. 3, 2011

(54) SURGICAL TOOL CALIBRATING DEVICE HAVING ELECTRONIC SENSING MODULE

(75) Inventors: Hung-Sheng Tien, Guishan Shiang (TW); Yen-Chu Chen, Guishan Shiang (TW); Jia-Jyun Hong, Guishan Shiang (TW); Chi-Bin Wu, Guishan Shiang (TW)

(73) Assignee: AccuMIS Inc., Zhonghe City (TW)

(21) Appl. No.: 12/577,767

(22) Filed: Oct. 13, 2009

(30) Foreign Application Priority Data
Aug. 26, 2009 (TW) 098128591

(51) Int. Cl. A61B 17/00

(52) U.S. Cl. 606/1

(57) ABSTRACT

A surgical tool calibrating device having an electronic sensing module is provided. The surgical tool calibrating device includes a calibration block, an electronic sensing module, and a signal communication module. The calibration block has at least one tool inserting portion, and the electronic sensing module is disposed on the bottom of the said tool inserting portion for generating an activating signal. The signal communication module is electrically connected to the electronic sensing module for transmitting the activating signal. With the electronic sensing module being provided in the calibration block, the activating signal is generated immediately when a surgical tool is inserted into the calibration block and makes contact with the electronic sensing module. Thus, the surgical tool calibrating device is activated in real time so as to determine the length of and the suitable bore diameter for the surgical tool.

### BACKGROUND OF THE INVENTION

1. Technical Field

The present invention relates to a surgical tool calibrating device. More particularly, the present invention relates to a surgical tool calibrating device which is applicable to medical image scanning and has an electronic sensing module for reducing errors in calibrating a surgical tool.

2. Description of Related Art

With continuous advancement in computer technology and rapid development of three-dimensional medical imaging techniques, surgical operations that require high positioning precision are now performed with the assistance of computers. Therefore, computer-assisted surgery is not only a major research field nowadays, but also an important trend in the future. For example, computers can be used to guide the surgical procedures of, among many others, neurosurgery in the brain, vertebral fixation and lumbar puncture, total knee replacement, and total hip joint replacement. However, all these surgical procedures demand very high precision and ample clinical experience; a slight error in the surgical path may injure the neighboring nerves. Hence, a computer-assisted surgical navigation system combines medical imaging with computer vision to help surgeons in performing high-precision surgical operations. Under such circumstances, it is of utmost importance to ensure preoperatively that surgical tools used in the computer-assisted surgical navigation system are precisely positioned.

FIG. 1 illustrates schematically a surgical tooling calibrating system 10 of a conventional surgical navigation system.

As shown in FIG. 1, the conventional surgical tool calibrating system 10 includes a calibration fixture 11, a calibration block 12, and an optical positioning device 13. The calibration fixture 11 is fixed in position to an end 21 of a surgical tool 20 and includes a plurality of first position indicating elements 111. On the other hand, the calibration block 12 has a side provided with a plurality of second position indicating elements 121. The calibration block 12 further has a side which is adjacent to the aforesaid side and formed with a plurality of calibration bores 122 to be inserted by a pointed end 22 of the surgical tool 20.

The optical positioning device 13 includes a scanner 131 and a signal processor 132. The scanner 131 is an infrared signal transceiver for transmitting an infrared signal (not shown). Meanwhile, the first and second position indicating elements 111 and 121 are provided respectively with reflective markers for reflecting the infrared signal. The scanner 131 receives the infrared signal reflected by the first and second position indicating elements 111 and 121 and thus obtains spatial coordinate information thereof. As the first and second position indicating elements 111 and 121 are so arranged as to have a fixed spatial relation therebetween, the signal processor 132 having received the spatial coordinate information transmitted from the scanner 131 can determine the actual dimension of the surgical tool 20 by triangulation, thereby calibrating dimensional information of the surgical tool 20.

In practice, the surgical tool 20 is inserted by an operator into one of the calibration bores 122 that has the appropriate bore diameter, and the surgical tool 20 is subsequently lowered to a specific depth. (The surgical tool 20 is usually inserted along the calibration bore 122 until the bottom of the calibration block 12 is reached.) Then, the operator manually selects a corresponding bore diameter shown on a screen and thus activates a calibration procedure of the conventional surgical tool calibrating system 10. However, calibration errors are bound to occur if the operator inadvertently inserts the surgical tool 20 into a calibration bore 122 whose bore diameter does not match the surgical tool 20, or if the operator fails to make sure that the surgical tool 20 reaches the specific depth, or if the operator activates the calibration procedure by selecting a wrong bore diameter on the screen. For obvious reasons, the operation described above tends to result in imprecise calibration and inconvenience of use.

### BRIEF SUMMARY OF THE INVENTION

It is an objective of the present invention to provide a surgical tool calibrating device having an electronic sensing module, wherein the electronic sensing module and a signal communication module are disposed on the bottom of a calibration block. Thus, when a pointed end of a surgical tool makes contact with the electronic sensing module, an activating signal is automatically generated. The activating signal is transmitted by the signal communication module to an optical positioning device so as to trigger a calibration procedure for the surgical tool in real time, thereby increasing the precision in calibrating the surgical tool.

It is another objective of the present invention to provide a surgical tool calibrating device having an electronic sensing module, wherein a calibration block is formed with at least one tool inserting portion, and each tool inserting portion is provided with a sensing unit on its bottom. Thus, when a pointed end of a surgical tool makes contact with the bottom of a certain tool inserting portion corresponding in dimension to the surgical tool and therefore makes contact with the corresponding sensing unit, an activating signal is automatically generated. The bore diameter of the tool inserting portion being inserted is immediately identified by an optical positioning device so as to prevent manual operation errors.

In order to achieve the above and other objectives, the present invention provides a surgical tool calibrating device having an electronic sensing module, wherein the surgical tool calibrating device includes: a calibration block having at least one tool inserting portion; the electronic sensing module provided on the bottom of at least one tool inserting portion and configured for generating an activating signal; and a signal communication module electrically connected to the electronic sensing module so as to transmit the activating signal.

Implementation of the present invention involves at least the following inventive steps:

1. With the electronic sensing module being provided on the bottom of the calibration block, and due to the property of a piezoelectric element provided in the electronic sensing module, the activating signal is generated as soon as a pointed end of a surgical tool makes contact with the electronic sensing module. The activating signal activates the surgical tool calibrating device and thereby allows the bore diameter of the tool inserting portion being inserted to be identified in real time.

2. When a surgical tool is inserted into the calibration block and makes contact with the electronic sensing module, the activating signal is immediately generated so as for the surgical tool calibrating device to trigger a calibration procedure for the surgical tool in real time and thereby obtain the length of and the appropriate bore diameter for the surgical tool. Consequently, manual operation errors are prevented, and precision in calibrating the surgical tool is effectively enhanced.

### BRIEF DESCRIPTION OF THE SEVERAL VIEWS OF THE DRAWINGS

A detailed description of further features and advantages of the present invention is given below so that a person skilled in the art can understand and implement the technical contents of the present invention and readily comprehend the objectives and advantages thereof by reviewing the teachings disclosed herein and the appended claims in conjunction with the accompanying drawings, in which:

FIG. 1 is a perspective view of a surgical tool calibrating system of a conventional surgical navigation system;

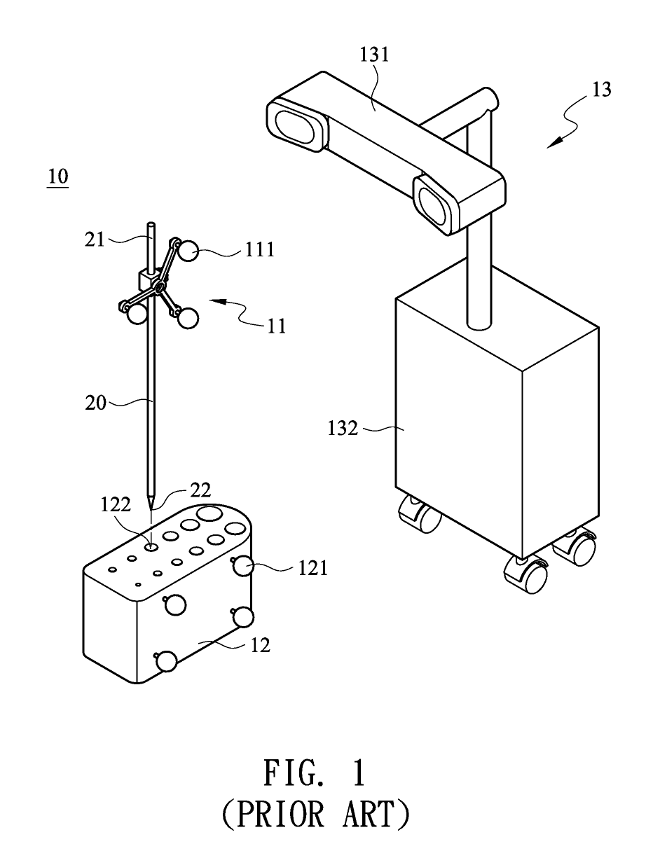

FIG. 2A is a perspective view of a surgical tool calibrating device having an electronic sensing module according to the present invention, wherein a surgical tool has yet to be inserted into a tool inserting portion of a calibration block;

FIG. 2B is another perspective view of the surgical tool calibrating device according to the present invention, wherein the surgical tool has been inserted into a tool inserting portion of the calibration block;

FIG. 2C is a detailed view of the calibration block shown in FIG. 2A, with the surgical tool inserted into the tool inserting portion;

FIG. 3 is a function block diagram of the surgical tool calibrating device according to the present invention; and

FIG. 4 is a flowchart of operation of the surgical tool calibrating device according to the present invention.

### DETAILED DESCRIPTION OF THE INVENTION

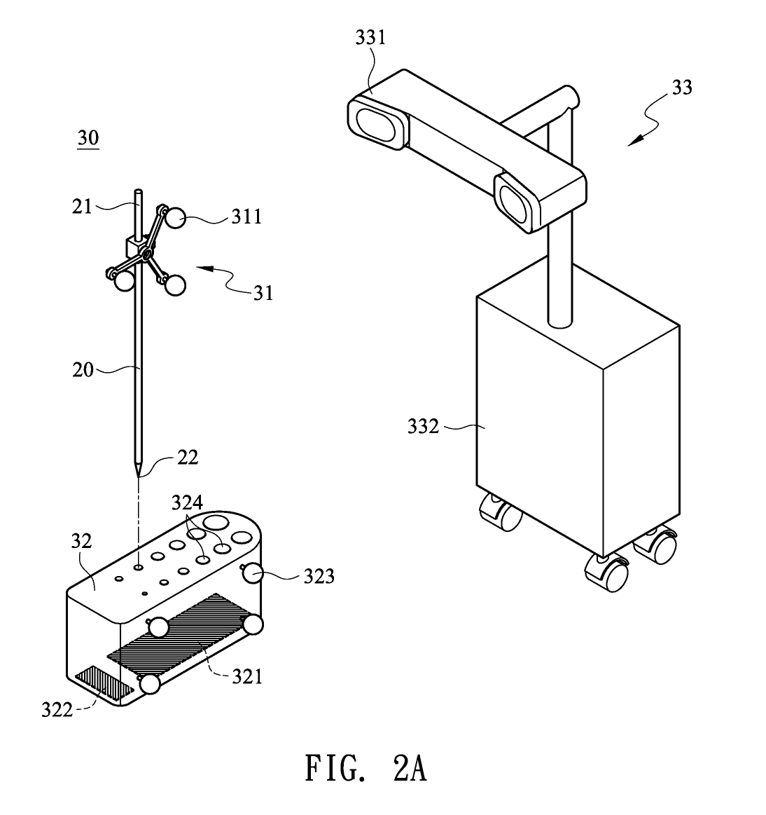

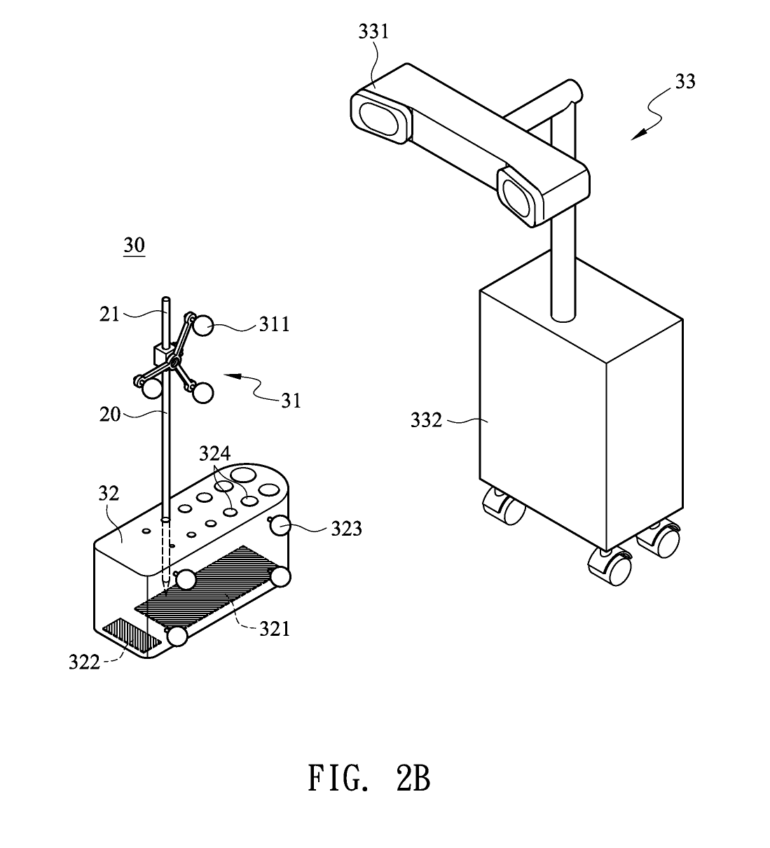

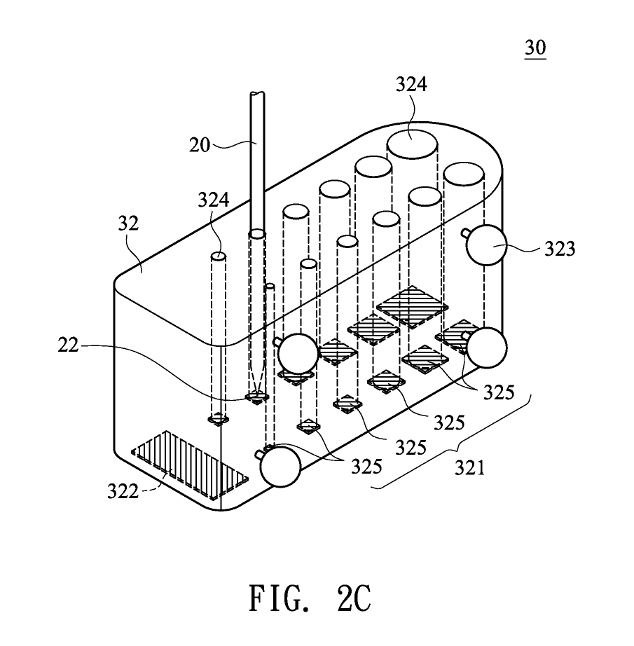

Referring to FIG. 2A and FIG. 2B, a surgical tool calibrating system 30 having an electronic sensing module essentially includes a calibration fixture 31, a surgical tool calibrating device, and an optical positioning device 33. According to an embodiment of the present invention, the surgical tool calibrating device includes a calibration block 32, an electronic sensing module 321, and a signal communication module 322.

As in the prior art, the calibration fixture 31 is fixed in position to an end 21 of a surgical tool 20 and includes a plurality of first position indicating elements 311.

Also as in the prior art, the calibration block 32 has a side provided with a plurality of second position indicating elements 323. The calibration block 32 further has a side which is adjacent and perpendicular to the aforesaid side and provided with at least one tool inserting portion 324 to be inserted by a pointed end 22 of the surgical tool 20. Each of the said tool inserting portion 324 is formed as a calibration bore having a different bore diameter. Therefore, as the dimension of the surgical tool 20 varies, one of the said tool inserting portion 324 can be selected for being inserted by the surgical tool 20, wherein the bore diameter of the said tool inserting portion 324 corresponds to the dimension of the surgical tool 20. Consequently, calibration errors which may otherwise result from failure to insert the surgical tool 20 to the specific depth are effectively prevented.

The electronic sensing module 321 is disposed on the bottom of the said tool inserting portion 324 while the signal communication module 322 is electrically connected to the electronic sensing module 321. When the surgical tool 20 is inserted into one of the said tool inserting portion 324 and reaches a specific depth, the pointed end 22 of the surgical tool 20 makes contact with the electronic sensing module 321, and the activating signal is generated as a result. The activating signal is sent to the signal communication module 322 for subsequent transmission. The signal communication module 322 is a wire-based communication module or a wireless communication module, so that the signal communication module 322 transmits the activating signal either wirelessly or via a wire.

Preferably, as shown in FIG. 2C, the electronic sensing module 321 includes a plurality of sensing units 325, wherein each sensing unit 325 includes a piezoelectric element. Furthermore, each sensing unit 325 corresponds in position to one of the said tool inserting portion 324. Therefore, when the surgical tool 20 is inserted into the said tool inserting portion 324, whose dimension corresponds to that of the surgical tool 20, the surgical tool 20 makes contact only with one of the corresponding sensing units 325. When the activating signal emitted by this corresponding sensing unit 325 reaches the signal communication module 322, the optical positioning device 33 activates a tool calibration procedure and thereby identifies the bore diameter of the said tool inserting portion 324 inserted by the surgical tool 20. Consequently, the selection of a wrong bore diameter due to manual operation errors is prevented.

The optical positioning device 33 includes a scanner 331 and a signal processor 332, wherein the scanner 331 is an infrared transceiver. In response to the activating signal transmitted by the signal communication module 322, the optical positioning device 33 begins an optical positioning process (e.g., triangulation) and carries out the calibration procedure for the surgical tool 20 in a way similar to the prior art, i.e., by sending an infrared signal and receiving a reflection thereof so as to determine the spatial coordinate information of each position indicating element. In order to reflect the infrared signal emitted by the scanner 331, the first and second position indicating elements 311, 323 each include a reflective marker, or the first and second position indicating elements 311, 323 are each a reflective ball or an infrared reflector.

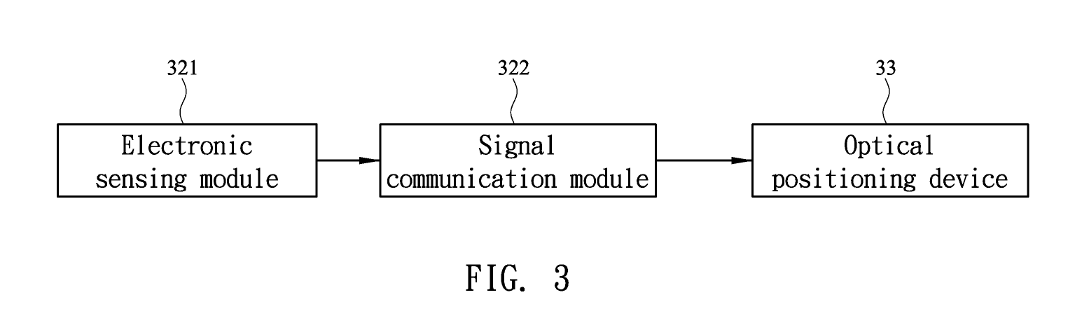

Please refer to FIG. 3 for a function block diagram of the present embodiment.

Specifically speaking, as the electronic sensing module 321 includes a piezoelectric element (not shown), when the pointed end 22 of the surgical tool 20 makes contact with the electronic sensing module 321, as shown in FIG. 2B, a contact pressure is generated. Thus, an electronic signal is produced and transmitted through the signal communication module 322 to the optical positioning device 33, wherein the electronic signal serves as the activating signal. After receiving the activating signal, the optical positioning device 33 activates the tool calibration procedure and, through subsequent detection and calculation, determines the actual length of and the appropriate bore diameter for the surgical tool 20, thereby calibrating dimensional information of the surgical tool 20.

As in the prior art, the first and second position indicating elements 311, 323 are so arranged as to have a fixed spatial relation therebetween. Therefore, the only variable in the tool calibration procedure is the length of the surgical tool 20, namely the distance between the first position indicating elements 311 and the pointed end 22 of the surgical tool 20. By implementation of the present embodiment, an operator can make sure that the surgical tool 20 is positioned at a specific position where the pointed end 22 of the surgical tool 20 makes contact with the bottom of the calibration block 32; moreover, the tool calibration procedure can be triggered in real time so as to increase the precision in calibrating the surgical tool 20.

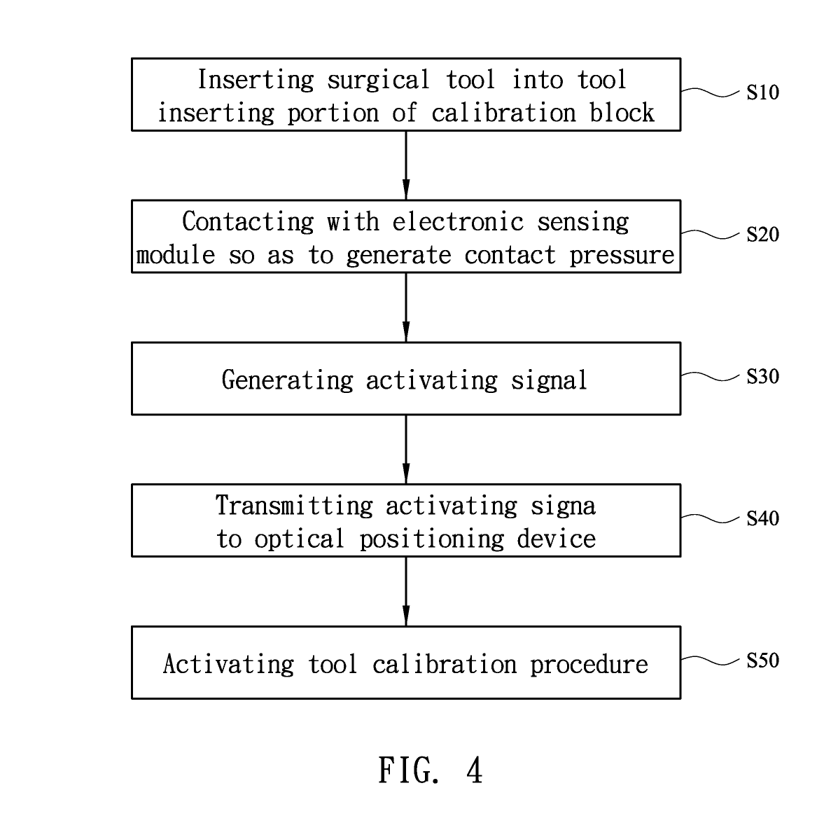

Please refer to FIG. 4 for a flowchart of operation of the present embodiment.

At step S10, an operator inserts a surgical tool into a certain tool inserting portion of the calibration block that corresponds in dimension to the surgical tool. At step S20, the surgical tool makes contact with the electronic sensing module and thus generates a contact pressure. Then, the activating signal is generated at step S30 and is transmitted by the signal communication module to the optical positioning device at step S40. Finally, the tool calibration procedure is activated in real time at step S50 according to the activating signal.

The foregoing embodiment is illustrative of the characteristics of the present invention so as to enable a person skilled in the art to gain insight into the contents disclosed herein and implement the present invention accordingly. The embodiment, however, is not intended to restrict the scope of the present invention. Hence, all equivalent modifications and variations which do not depart from the spirit and principle of the present invention should fall within the scope of the appended claims.

What is claimed is:

1. A surgical tool calibrating device having an electronic sensing module, the surgical tool calibrating device comprising:
a calibration block having at least one tool inserting portion;
the electronic sensing module provided on the bottom of the said tool inserting portion and configured for generating an activating signal; and
a signal communication module electrically connected to the electronic sensing module so as to transmit the activating signal.

2. The surgical tool calibrating device of claim 1, wherein the calibration block comprises a plurality of first position indicating elements.

3. The surgical tool calibrating device of claim 2, wherein each said first position indicating element comprises a reflective marker.

4. The surgical tool calibrating device of claim 2, wherein each said first position indicating element is a reflective ball.

5. The surgical tool calibrating device of claim 2, wherein each said first position indicating element is an infrared reflector.

6. The surgical tool calibrating device of claim 1, wherein each said tool inserting portion is a calibration bore.

7. The surgical tool calibrating device of claim 1, wherein the calibration block has a plurality of said tool inserting portions, and each said tool inserting portion is a calibration bore.

8. The surgical tool calibrating device of claim 7, wherein the calibration bores have different bore diameters.

9. The surgical tool calibrating device of claim 7, wherein the electronic sensing module comprises a plurality of sensing units, and each said sensing unit is provided on the bottom of the corresponding said tool inserting portion.

10. The surgical tool calibrating device of claim 9, wherein each said sensing unit comprises a piezoelectric element.

11. The surgical tool calibrating device of claim 1, wherein the electronic sensing module comprises a piezoelectric element.

12. The surgical tool calibrating device of claim 1, wherein the signal communication module is a wire-based communication module or a wireless communication module.
